(19) United States
(12) Patent Application Publication
(10) Pub. No.: US 2009/0018383 A1
(43) Pub. Date: Jan. 15, 2009

(54) EXPANDABLE BRACHYTHERAPY DEVICE WITH EXPANDABLE CAGE HAVING A CONTROLLED DIAMETER

(75) Inventors: Mia Michelle Corcione, Pleasanton, CA (US); Darrell Drysen, Altadena, CA (US); Fredrick W. Wintch, Snohomish, WA (US)

Correspondence Address: MCDERMOTT WILL & EMERY LLP, 2049 CENTURY PARK EAST, 38th Floor, LOS ANGELES, CA, 90067-3208, US

(73) Assignee: NORTH AMERICAN SCIENTIFIC, INC., Chatsworth, CA (US)

(21) Appl. No.: 12/137,437

(22) Filed: Jun. 11, 2008

Related U.S. Application Data
(63) Continuation-in-part of application No. 11/737,028, filed on Apr. 18, 2007, now Pat. No. 7,357,770.
(60) Provisional application No. 60/882,391, filed on Dec. 28, 2006.
(60) Provisional application No. 60/864,288, filed on Nov. 3, 2006.

(51) Int. Cl. A61M 36/04

(52) U.S. Cl. 600/7

(57) ABSTRACT

A brachytherapy device may include an expandable outer cage an expandable inner cage positioned within the outer cage and configured to receive radioactive material at its perimeter. The expandable inner cage may be configured to expand, and the outer cage may be configured to expand to a variable diameter greater than the inner cage diameter so as to fill resected cavities of various diameters.

### CROSS-REFERENCE TO RELATED APPLICATION(S)

This application is a continuation in part of patent application Ser. No. 11/737,028, filed Apr. 18, 2007, entitled “EXPANDABLE BRACHYTHERAPY DEVICE WITH CONSTANT RADIATION SOURCE SPACING,” attorney docket no. 63344-050, which is based upon and claims the benefit of U.S. Provisional Patent Application Ser. No. 60/882,391, entitled “EXPANDABLE BRACHYTHERAPY DEVICE WITH CONSTANT RADIATION SOURCE SPACING,” filed Dec. 28, 2006, attorney docket no. 63344-048. The entire content of both applications is incorporated herein by reference.

This application is related to U.S. Provisional Patent Application Ser. No. 60/864,288, entitled “BRACHYTHERAPY DEVICE HAVING SEED TUBES WITH INDIVIDUALLY-SETTABLE TISSUE SPACINGS,” filed Nov. 3, 2006, attorney docket no. 63344-045. This application is also related to U.S. patent application Ser. Nos. 11/305,437, entitled “BRACHYTHERAPY APPARATUS,” filed Dec. 16, 2005, attorney docket no. 63344-021, and Ser. No. 11/379,739, entitled “BRACHYTHERAPY APPARATUS FOR ASYMMETRICAL CAVITIES,” filed Apr. 21, 2006, attorney docket no. 63344-023. This application is also related to patent application Ser. No. 11/737,028, filed Apr. 18, 2007, entitled “EXPANDABLE BRACHYTHERAPY DEVICE WITH CONSTANT RADIATION SOURCE SPACING,” attorney docket no. 63344-050, which is based upon and claims the benefit of U.S. Provisional Patent Application Ser. No. 60/882,391, entitled “EXPANDABLE BRACHYTHERAPY DEVICE WITH CONSTANT RADIATION SOURCE SPACING,” filed Dec. 28, 2006, attorney docket no. 63344-048. The entire content of all three of these applications is incorporated herein by reference.

### BACKGROUND

1. Field

This application relates to brachytherapy.

2. Description of Related Art

Brachytherapy applies radiation to tissue by placing the source of radiation close to the tissue. Oftentimes, a high dose of radiation is needed. However, it may be difficult to apply a high dose to areas in need of treatment using brachytherapy, without also causing damage to healthy tissue in the vicinity.

One approach to addressing this difficulty is to utilize seed tubes with individually-settable tissue spacings, as described in U.S. Provisional Application Ser. No. 60/864,288, entitled “BRACHYTHERAPY DEVICE HAVING SEED TUBES WITH INDIVIDUALLY-SETTABLE TISSUE SPACINGS,” filed Nov. 3, 2006, attorney docket number 63344-045. The technician may individually set the separation distance between each seed tube that carries a radiation seed and the wall of the cavity in which it is placed.

Expertise may be required to ascertain and set the desired spacings in such a device. The use of different spacings, moreover, may complicate the process of creating and implementing an effective treatment regimen.

### SUMMARY

A brachytherapy device may include an expandable outer cage configured to variably expand so as to substantially fill a resected cavity; an expandable inner cage may be positioned and configured to expand within the outer cage and may be configured to receive radioactive material at its perimeter. The outer cage may expand to variable diameters and the inner cage may expand simultaneously and at a variable diameter which may be less than the outer cage diameter.

The inner and outer cages may be expanded via rotatable thumb wheel which may engage a rack and pinion mechanism. The brachytherapy device may include a rod running through the outer and inner cages that is attached to the handle at a distal end.

Alternatively, a brachytherapy device may include an expandable outer cage, an expandable inner cage positioned within the outer cage and configured to receive radioactive material at its perimeter, the inner cage may expand to a fixed diameter, while the outer cage may expand to a variable diameter to fill resected cavities.

The outer cage may be expanded via a rotatable thumb wheel which may engage a rack and pinion gear mechanism and the inner cage may expand under the force of a compression spring.

The brachytherapy device may include a handle in which the rotatable thumb wheel, rack and pinion and spring move. The brachytherapy device may include a rod running through the outer and inner cages that is attached to the handle at a distal end.

The outer and inner cages may each include a plurality of tubes. Each of the tubes and the rod may have a proximal end. The proximal end of all of the tubes and the rod may be affixed to one another. Each of the tubes may have a distal end and the actuator may be configured to engage the distal ends of the tubes. The actuator may be configured to apply longitudinal compressive force to the distal ends of the tubes. The outer and inner cages may be configured to collapse into a rod-like shape when the distal ends of the tubes are not engaged by the thumb wheel or the compressive spring.

The inner and outer cages may include hollow tubes. The hollow inner tubes may be configured to receive radioactive material at different locations therein.

Each of the hollow outer and inner tubes may be configured to have hollow lumens and a super-elastic alloy insert may be inserted into the lumen of all or some of the hollow lumen tubes. As used herein, a super-elastic alloy belong to the larger family of shape memory alloys and exhibit an elastic (impermanent) response to relatively high stress. This super-elastic alloy insert may be Nitinol and may also be of a material that is visible to imaging technology, thus causing the shape of the cage to be visible during a diagnostic scan, and may provide a check on the adequacy of the cage expansion.

A brachytherapy device may include a plurality of flexible outer tubes, each having a first length and a distal end. The device may include a plurality of flexible inner tubes positioned within a volume defined by the outer tubes, each having a second length different from the first length, each being configured to receive radioactive material, and each having a distal end. The device may include a movable actuator configured to engage the distal ends of the outer and the inner tubes.

The distal ends of the outer tubes may lie in a first plane and the distal ends of the inner tubes may lie in a second plane. The first and the second planes may be parallel and separated from one another.

### BRIEF DESCRIPTION OF DRAWINGS

The drawings disclose illustrative embodiments. They do not set forth all embodiments. Other embodiments may be used in addition or instead. Details that may be apparent or unnecessary may be omitted to save space or for more effective illustration. When the same numeral appears in different drawings, it is intended to refer to the same or like components or steps.

FIG. 1A illustrates a brachytherapy device in a fully collapsed position.

FIG. 1B illustrates the brachytherapy device illustrated in FIG. 1A after an outer cage has been partially expanded to a first diameter and the inner cage has been expanded to a smaller diameter than the outer cage.

FIG. 1C illustrates the brachytherapy device illustrated in FIG. 1 after an outer cage has been further expanded to a second larger diameter and the inner cage has been further expanded to a second larger diameter which is smaller than the diameter of the outer cage.

FIG. 1D illustrates the brachytherapy device illustrated in FIG. 1 after an outer cage has been further expanded to a third larger diameter and the inner cage has been expanded to a third larger diameter which is smaller than the diameter of the outer cage.

FIG. 2A illustrates a cross section view of FIG. 1A.

FIG. 2B illustrates a cross section view of FIG. 1B.

FIG. 2C illustrates a cross section view of FIG. 1C.

FIG. 2D illustrates a cross section view of FIG. 1D.

FIG. 3 is a cross-section of the tubes illustrated in FIGS. 1A and 4A in a collapsed state.

FIG. 3A is a cross-section of the tubes of FIGS. 1A and 4A in a collapsed state showing super-elastic wires inserted into the hollow lumen of the outer tubes.

FIG. 4 is an exploded perspective view of FIG. 1A.

FIG. 5 is a partially exploded view of FIG. 1A.

FIG. 6A illustrates an alternate brachytherapy device in a fully collapsed position.

FIG. 6B illustrates the brachytherapy device illustrated in FIG. 4A after an outer cage has been partially expanded to a first diameter and the inner cage has been expanded to a fixed diameter.

FIG. 6C illustrates the brachytherapy device illustrated in FIG. 4A after an outer cage has been further expanded to a second larger diameter and the inner cage has been expanded to a fixed diameter.

FIG. 6D illustrates the brachytherapy device illustrated in FIG. 4C after an outer cage has been further expanded to a third larger diameter and the inner cage has been expanded to a fixed diameter.

FIG. 7A illustrates a cross section view of FIG. 6A.

FIG. 7B illustrates a cross section view of FIG. 6B.

FIG. 7C illustrates a cross section view of FIG. 6C.

FIG. 7D illustrates a cross section view of FIG. 6D.

FIG. 8 is a cross-sectional view of FIG. 6A.

FIG. 9 illustrates stop screws on the handle illustrated in FIG. 6A.

### DETAILED DESCRIPTION OF ILLUSTRATIVE EMBODIMENTS

Illustrative embodiments are now discussed. Other embodiments may be used in addition or instead. Details that may be apparent or unnecessary may be omitted to save space or for a more effective presentation.

Brachytherapy devices may be used to treat cancerous tissue. Examples of such devices and ways in which they may be used are set forth in U.S. Provisional Patent Application 60/864,288, entitled “BRACHYTHERAPY DEVICE HAVING SEED TUBES WITH INDIVIDUALLY-SETTABLE TISSUE SPACINGS,” filed Nov. 3, 2006, attorney docket number 63344-045, the entire content of which is incorporated herein by reference. Except for differences described in this utility application, the brachytherapy devices and associated apparatus that are described in the aforementioned provisional patent application, and the ways in which they may be used, may be used in whole or in part in connection with the devices that are described in this application.

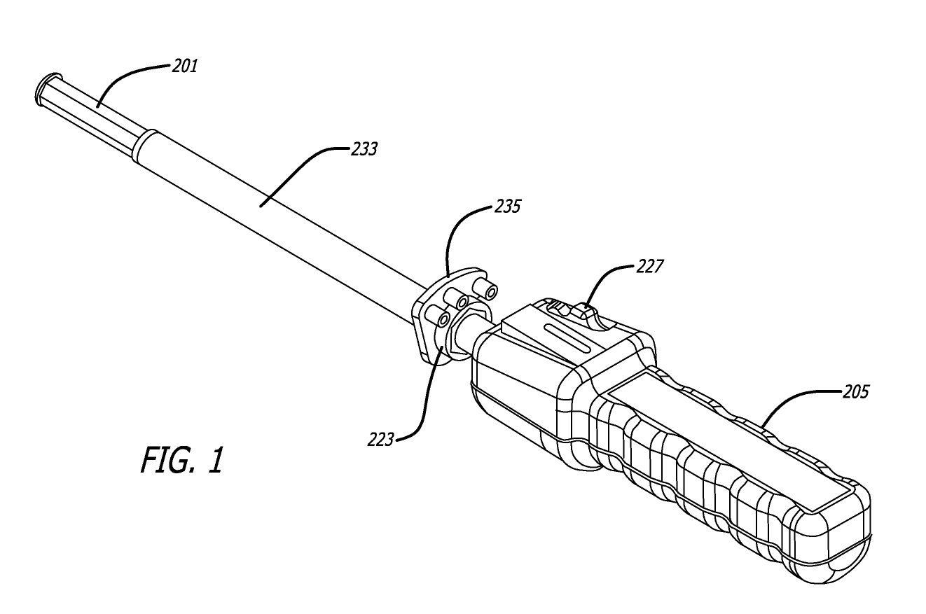

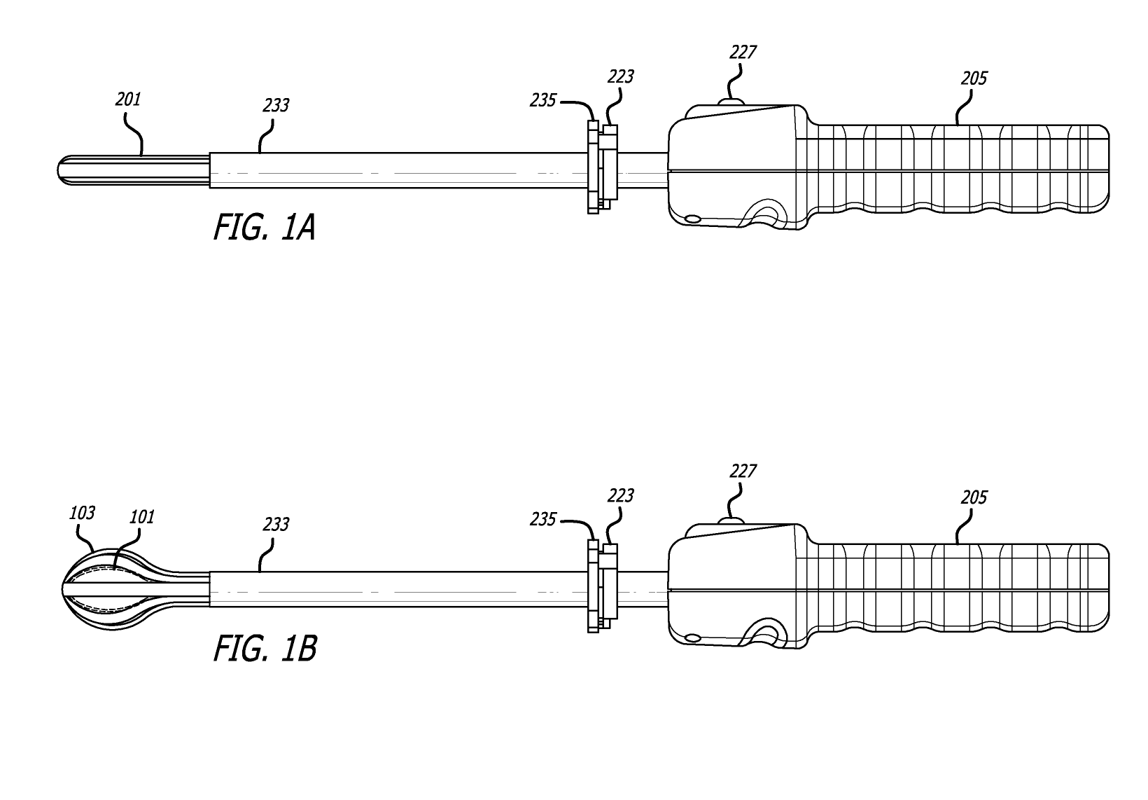

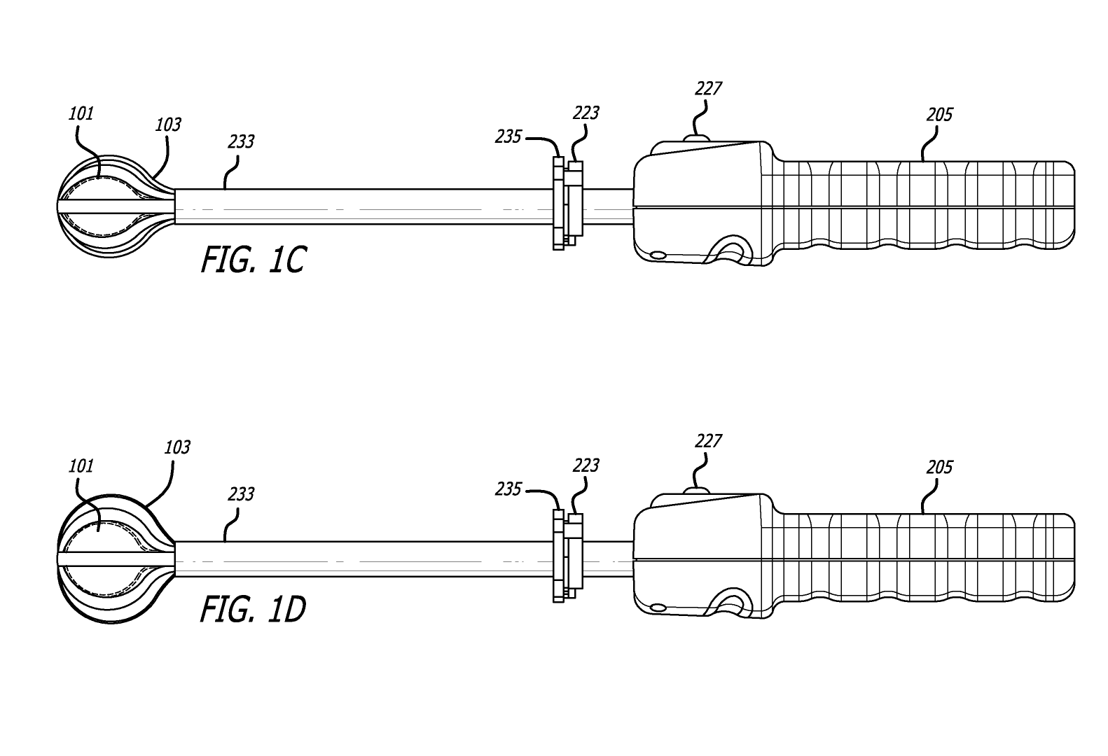

FIG. 1A illustrates a brachytherapy device in a fully collapsed position. As shown in FIG. 1A, the brachytherapy device may include tubes 201 positioned around a central rod 203 and attached to a handle 205. The handle 205 may include top and bottom part which may be connected via handle screws 207. The tubes 201 and the central rod 203 may be attached to one another at a proximal end 215 of the device away from handle 205 of the tubes 201 and the central rod 203. The tubes 201 may be covered in a sleeve 233 which may include a ring lock 235 and flange lock 223 on a distal end of the sleeve 233. The handle 205 may include a thumb wheel 227 which may protrude from inside the handle 205. The may include inner tubes 241 and/or outer tubes 221.

The brachytherapy device illustrated in FIG. 1A may be used in many ways. For example, a tumor may be removed from a breast of a patient. The distance between the surface of the breast and the entryway to the cavity may be measured. A sleeve having a length approximately equal to this measurement may be inserted through an incision in the breast until it reaches the entryway to the cavity. The sleeve may include an external flange that may be sutured to the skin of the breast. Details about illustrative apparatuses and processes that may be used are described in United States Provisional patent application entitled “BRACHYTHERAPY DEVICE HAVING SEED TUBES WITH INDIVIDUALLY-SETTABLE TISSUE SPACINGS,” Ser. No. 60/864,288, filed Nov. 3, 2006, attorney docket number 63344-045, the entire content of which is incorporated herein by reference.

The proximal end 215 of the collapsed brachytherapy device may be inserted into the sleeve 233 until the proximal end 215 passes through the entryway to the cavity and comes into contact with the opposite wall of the cavity.

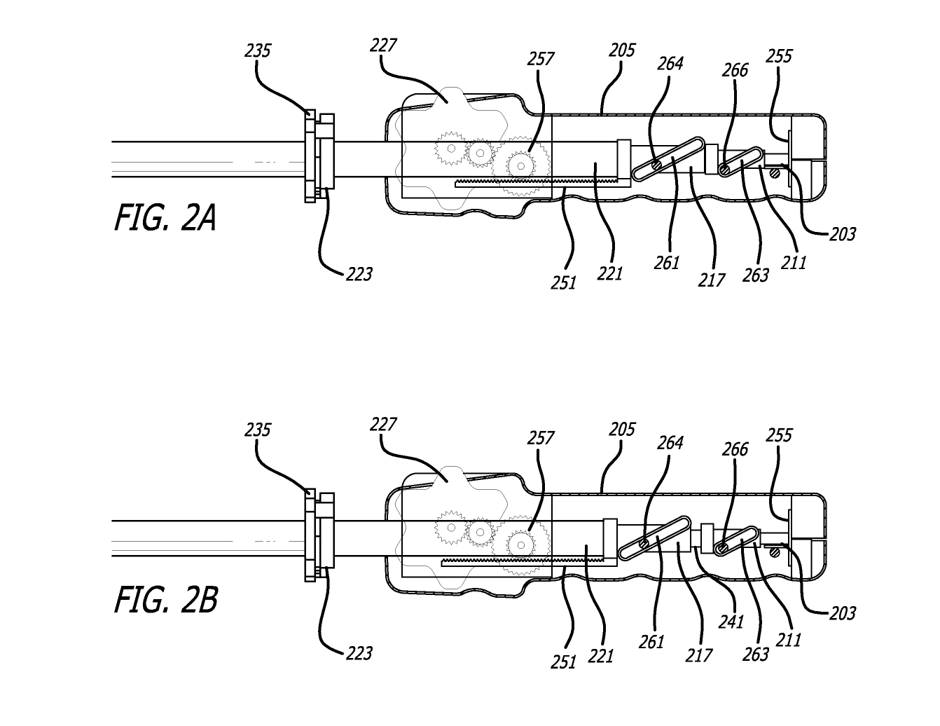

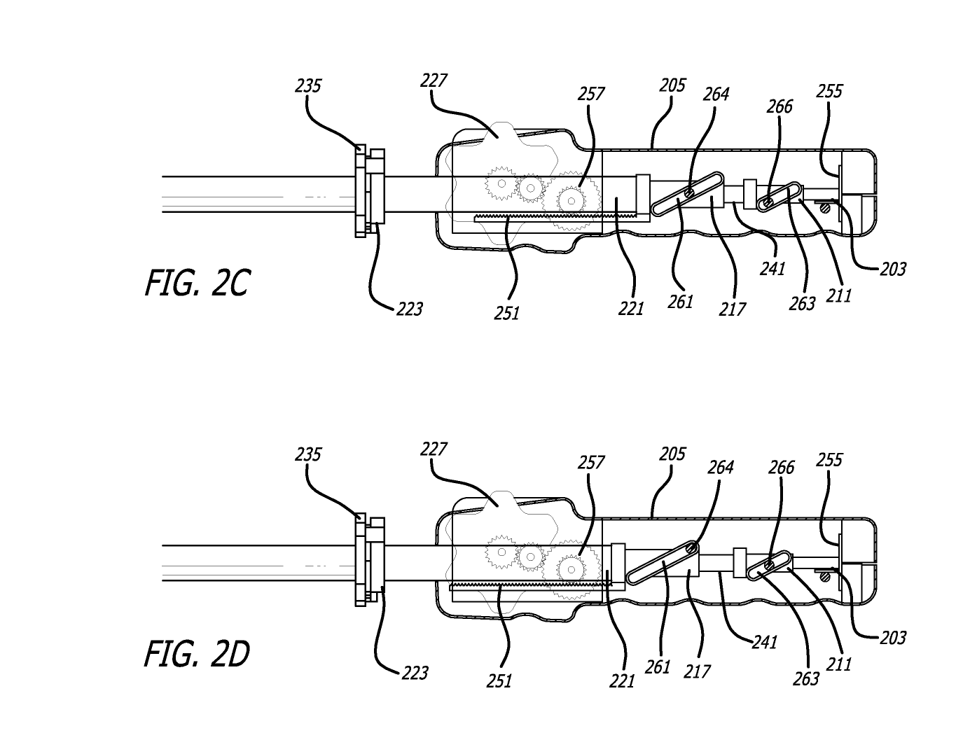

FIG. 1B illustrates the brachytherapy device illustrated in FIG. 1A after an outer cage 103 has been partially expanded to a first diameter and the inner cage 101 has been simultaneously partially expanded to a first diameter which may be smaller than the diameter of the outer cage 103. To expand the cages 101 and 103, the thumb wheel 227 may have been rotated with respect to the handle 205. This may have caused one or more gears 257 to rotate, as illustrated in FIG. 2B. The rotation of the gear 257 in turn, may have caused the longitudinal movement of the rack 251 in a proximal direction. This translation of the rotational movement of the thumb wheel to the longitudinal movement of the rack, may have subsequently caused the longitudinal movement of an inner cage carrier 211 and outer cage carrier 217, which may have compressed inner tubes 241 and outer tubes 221 of the tubes 201 respectively, thus causing them to bow, as illustrated in FIGS. 2B-2D.

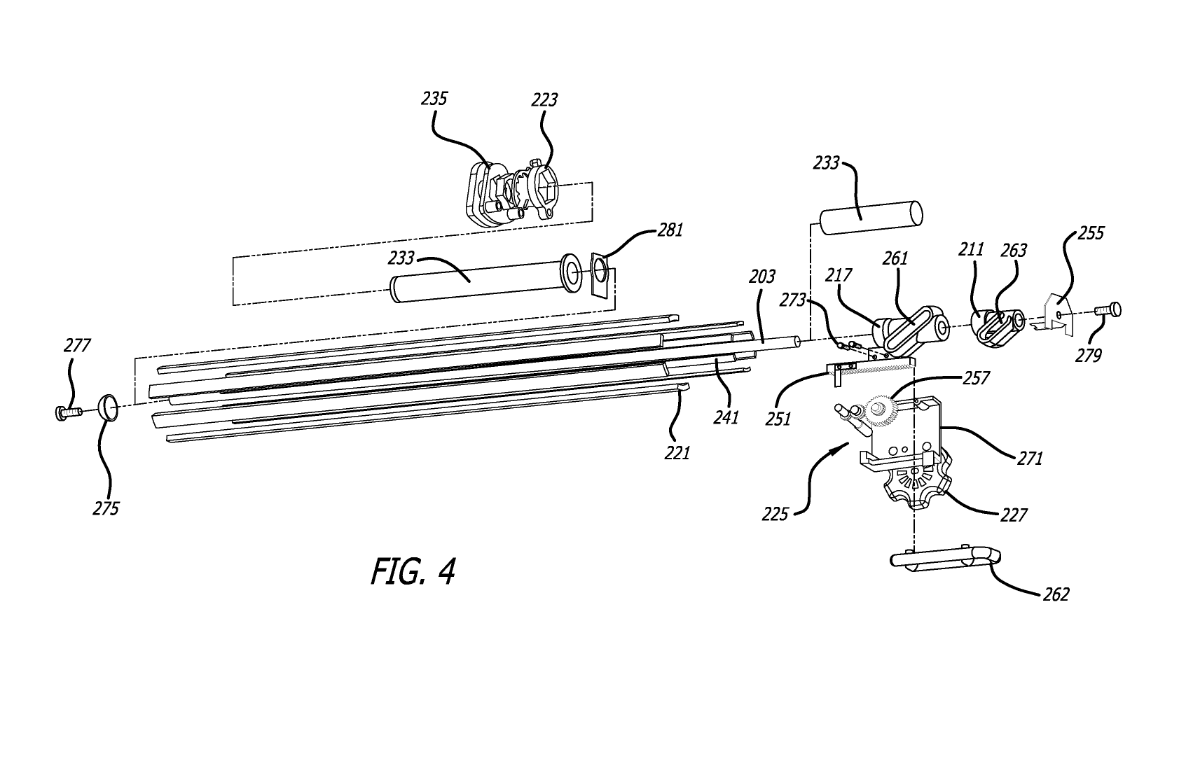

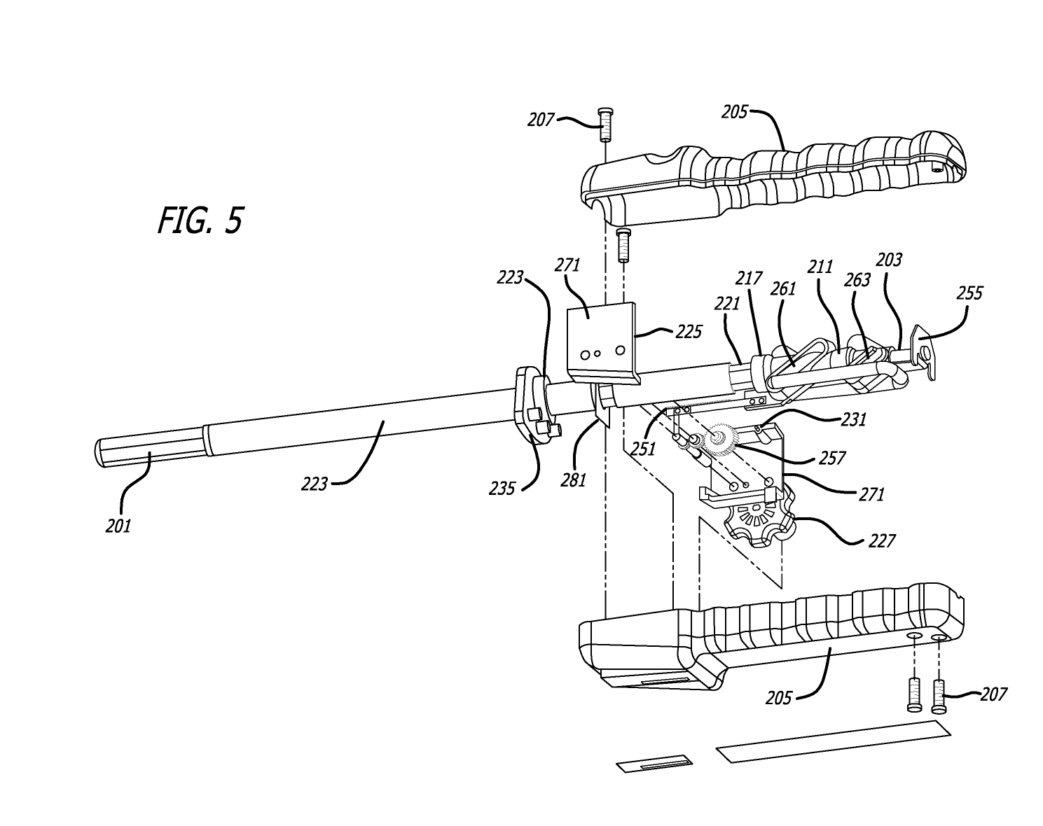

As partially shown in FIG. 4, the inner 241 and outer tubes 221 may be attached together at the proximal end of the device via end cap 275 and end screw 277.

As discussed above, the tubes 201 of the brachytherapy device may be within a sleeve 233 that has been inserted into the breast. As also explained above, the tubes 201 may protrude beyond the sleeve 233 at the entryway to the cavity until their proximal end 215 reaches the other wall of the cavity. In this configuration, only the portion of the tubes that lie within the cavity are free to bow.

Sleeve 233 may include end plate 281 between a distal end of sleeve and proximal wall of gear box 225. The end plate 281 may maintain the sleeve 233 from entering the gear box cover 225.

As partially illustrated in FIG. 2A, inner tubes 241 defining an inner cage 101 may be contained within the volume defined by the outer tubes 221 and may remain uncompressed at this point and thus in an unbowed and collapsed state.

The inner tubes 241 and outer tubes 221 may be configured to have hollow lumens and a super-elastic insert 204 may be inserted into the lumen of all or some of the hollow lumen tubes. This super-elastic insert may be a wire, and the wire may be of Nitinol. Each of the hollow outer and inner tubes may be configured to have hollow lumens and a super-elastic alloy insert may be inserted into the lumen of all or some of the hollow lumen tubes. As used herein, a super-elastic alloy belong to the larger family of shape memory alloys and exhibit an elastic (impermanent) response to relatively high stress. This super-elastic alloy insert may be Nitinol. The insert may also be of a material that is visible to imaging technology, thus causing the shape of the cages 101, and or 103 to show up during a diagnostic scan, and may provide a check on the adequacy of the cage 101 and or 103 expansion.

As shown in FIGS. 2B-2D, the rotation of the thumb wheel 227 may cause the inner 241 and outer 221 tubes to bow via a rack and gear pinion mechanism. The rotation of the thumb wheel 227 by a user may in turn rotate the gear 257, which may in turn cause the longitudinal movement of the rack 251, thereby causing movement of hair pin connecter 262 via pins 264 and 266 along angular slots 261 of outer cage carrier 217 and slots 263 of inner cage carrier 211 respectively. This may result in compression and bowing of the inner 101 and 103 outer cages which may expand the inner 101 cage to a first diameter and the outer cage 103 to a first diameter which may be greater than the first diameter of the inner cage 101. Outer cage carrier 217 may be attached to rack 251 via rack screws 273. The proximal end of the hair pin connector 262 may be attached to the handle 205 via a horizontal portion of bracket 255, which may allow the hair pin connector 262 to engage slots 261 and 263. A perpendicular portion of the bracket 255 may secure central rod 203 via bracket end screw 279. The gear 257 and a portion of the rack 251 may be covered by gear box cover 225 which may include a front and back wall 271 and may be attached to the handle 205 by gear box screws 231.

Angular slots 261 of outer carrier 217 may be at the same angle as angular slots 263 of inner carrier and angular slots 261 may be greater in length than angular slots 263.

Thumb wheel 227 may include a spring in the interior of thumb wheel 227, the thumb wheel 227, may include an axle with a gear. The thumb wheel 227 gear may engage another gear 257 which may engage rack 251.

Further rotation of the thumb wheel 227, as shown in FIG. 2C may cause further expansion of each of the inner and outer cages 101 and 103 simultaneously to another second diameter, each second diameter may be larger than the first diameter and the diameter of the outer cage 103 may be greater than the diameter of the inner cage 101.

Subsequent rotation of the thumb wheel 227, as shown in FIG. 2D may cause further expansion of both the inner 101 and outer 103 cages simultaneously to another diameter, each diameter may be larger than the first and second diameters, and the diameter of the outer cage 103 may be greater than the diameter of the inner cage 101. The diameter of the inner 101 and outer cages 103 may be variably expanded or collapsed via forward and reverse rotation of the thumb wheel 327, and the diameter of the outer cage 103 may be greater than the diameter of the inner cage 101.

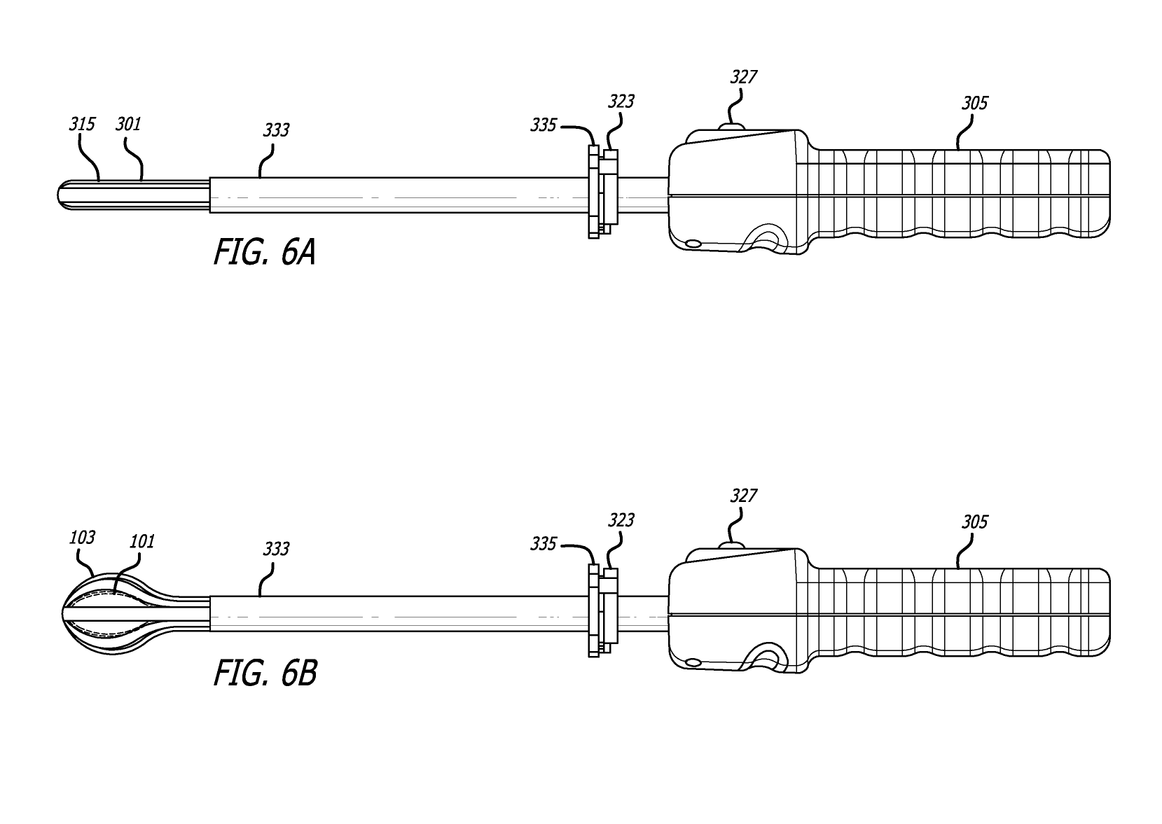

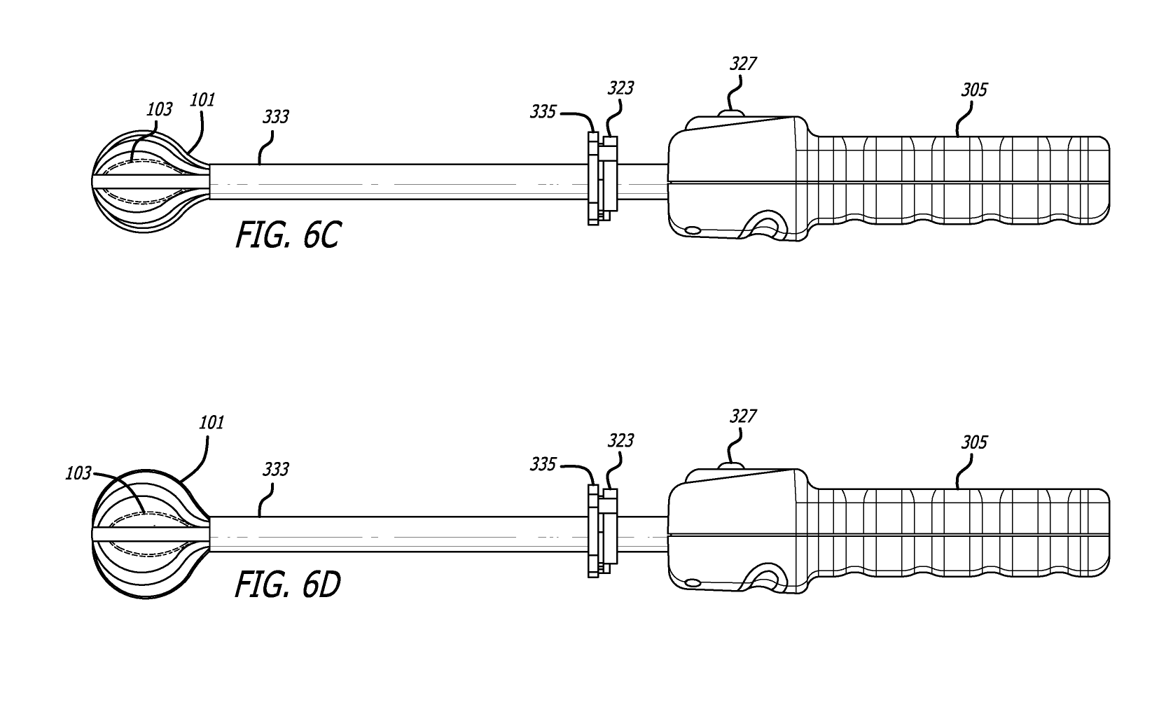

In an alternate embodiment, as illustrated in FIG. 6A, a brachytherapy device may include tubes 301 positioned around a central rod 303 and attached to a handle 305. The tubes 301 and the central rod 303 may be attached to one another at a proximal end 315 of the tubes 301 and the central rod 303. The tubes may be covered in a sleeve 333 which may include a ring lock 335 and flange lock 323 on a distal end of the sleeve 333. The handle 305 may include a thumb wheel 327 which may protrude from inside the handle 305.

The brachytherapy device illustrated in FIG. 6A may be used in the same way as the device as illustrated in FIG. 1A.

The proximal end 315 of the collapsed brachytherapy device may be inserted into the sleeve 333 until the proximal end 315 passes through the entryway to the cavity and comes into contact with the opposite wall of the cavity.

As discussed above, the tubes 301 of the brachytherapy device may be within a sleeve 333 that has been inserted into the breast. As also explained above, the tubes 301 may protrude beyond the sleeve at the entryway to the cavity until their proximal end 315 reaches the other wall of the cavity. In this configuration, only the portion of the tubes that lie within the cavity are free to bow and expand.

As partially illustrated in FIG. 6A, inner tubes 341 defining an inner cage 101 may be contained within the volume defined by the outer tubes 321 and may remain uncompressed at this point and thus in an unbowed and collapsed state.

The inner tubes 341 and outer tubes 321 may be configured to have hollow lumens and a super-elastic insert 204 may be inserted into the lumen of all or some of the hollow lumen tubes. As used herein, a super-elastic alloy belong to the larger family of shape memory alloys and exhibit an elastic (impermanent) response to relatively high stress. This super-elastic alloy insert may be Nitinol. This super-elastic insert may be a wire, and the wire may be of Nitinol. The insert may also be of a material that is visible to imaging technology, thus causing the shape of the cages 101, and or 103 to show up during a diagnostic scan, and may provide a check on the adequacy of the cage 101 and or 103 expansion.

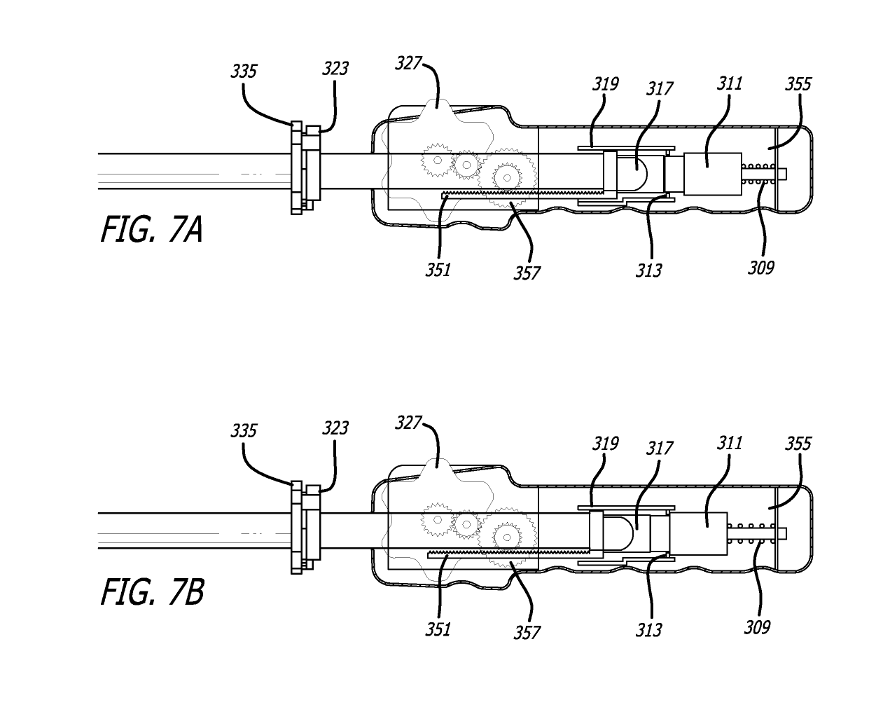

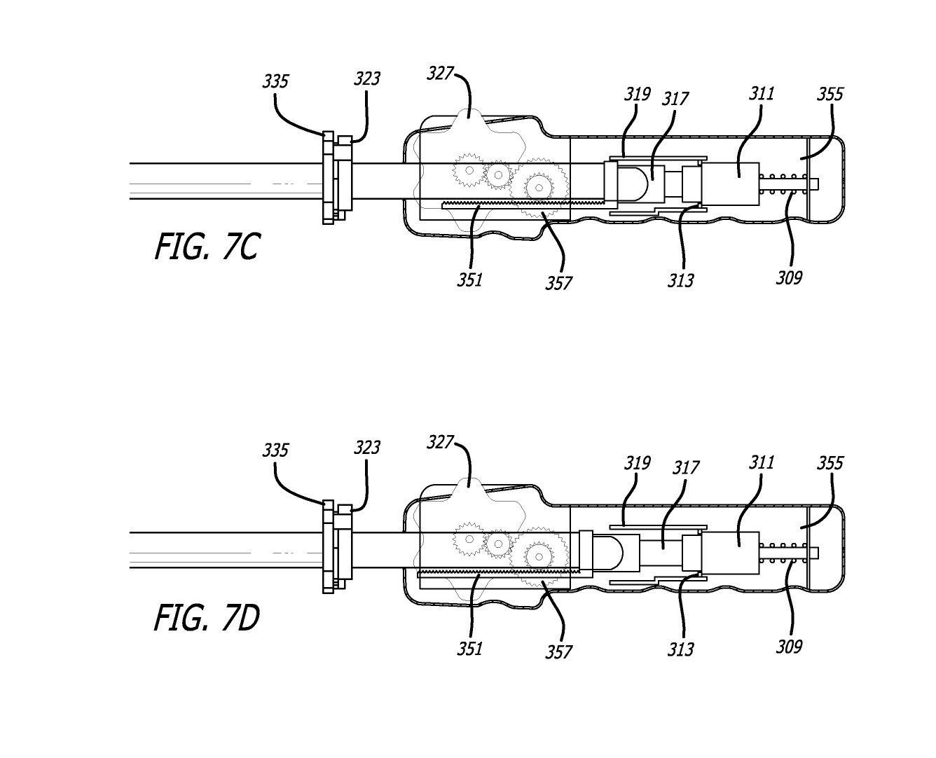

FIG. 6B illustrates the brachytherapy device illustrated in FIG. 6A after an outer cage 103 has been partially expanded to a first diameter and the inner cage 101 has been simultaneously expanded to a fixed diameter which may be substantially the same diameter as the outer cage 103. To expand the cages 101 and 103 into this position, the thumb wheel 327 may have been rotated with respect to the handle 305. This may have caused one or more gears 357 to rotate, as illustrated in FIG. 7B.

The rotation of the one or more gears 357 in turn, may have caused the longitudinal movement of the rack 351 in a proximal direction. This translation of the rotational movement of the thumb wheel 327 to the longitudinal movement of the rack 351, may have subsequently caused the longitudinal movement of an outer cage carrier 317, which may have compressed outer tubes 321 of the tubes 301 and released compressed spring 309.

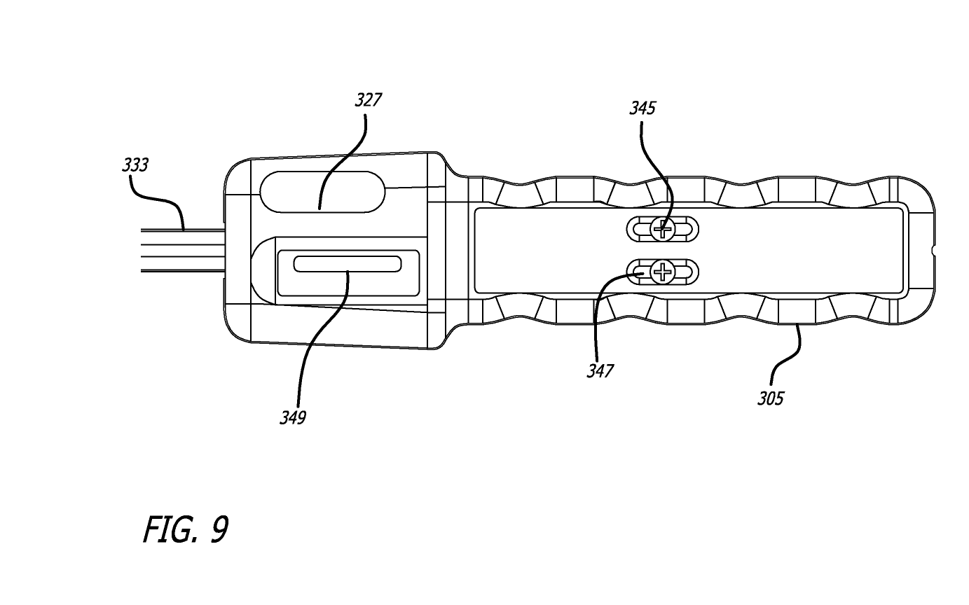

The spring 309 may be inserted over central rod 303 which in turn may be attached to bracket 355 via an end screw. The stop 313 may be adjustably arranged along handle 305 by stop lock screws 345 which may be inserted into stop lock screw slots 347, as shown in FIG. 9. The gears may be seen through slot 349 in handle 305.

The extension of spring 309 may have caused inner cage carrier 311 to moved along a distal direction until it engaged an adjustable stop 313. The movement of the inner cage carrier 311 may have compressed inner tubes 341 and may have caused them to bow and expand the inner cage 101 to substantially the same diameter as the outer cage 103, as illustrated in FIGS. 6B and 7B.

Subsequent rotation of thumb wheel 327, as shown in FIGS. 6C and 7C may cause the rack 351 to move further in the distal direction and may further cause the outer tube carrier 317 to move along guide system 319 and compress outer tubes 321. This may cause the outer cage 103 to further expand while the inner cage 101 remains at a fixed diameter.

Further rotation of the thumb wheel 327, as shown in FIGS. 6D and 7D may cause further expansion of the outer 103 cages to another diameter, while the inner cage 101 may remain at the fixed diameter following the initial thumb wheel 327 rotation. The diameter of the outer cages 103 may be variably expanded or collapsed via forward and reverse rotation of the thumb wheel 327, while the inner cage 101 may remain at the fixed diameter following the initial thumb wheel 327 rotation.

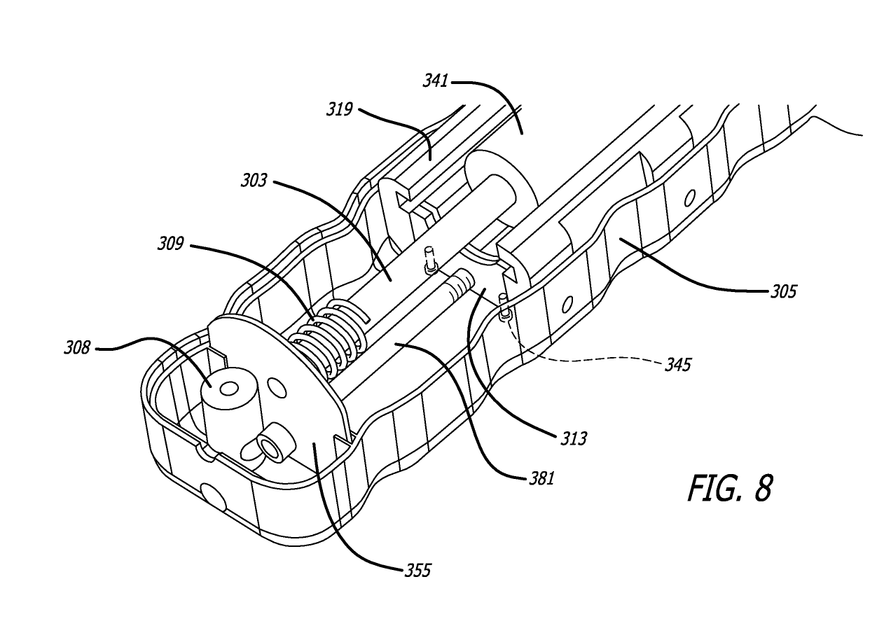

FIG. 8 shows a lead screw 381 may be inserted through bracket 305 parallel to central rod 303 to engage stop 313. The lead screw may be configured to fine tune the position of the stop 313, which in turn may set the inner cage 101 to fixed deployment diameter. Handle hole 308 may be arranged at a distal end of handle 305 to attach front and back sides of handle 305.

Thumb wheel 327 may include a spring 329 in the interior of thumb wheel 327, the thumb wheel 327, may include an axle with a gear. The thumb wheel 327 gear may engage another gear 357 which may engage rack 351.

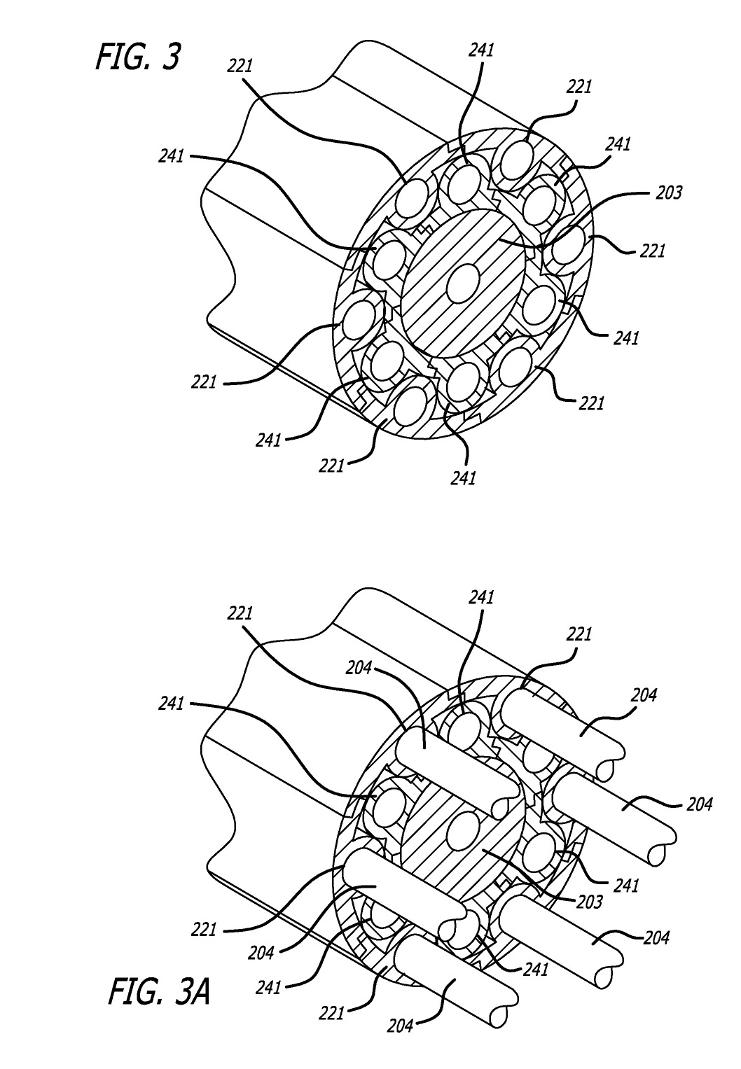

FIG. 3 is a cross-section of the tubes illustrated in FIG. 1A or 6A in a collapsed state. As shown in FIG. 3, the outer tubes 201 may define an interior volume in which the inner tubes 203 reside. Similarly, the inner tubes 241, 341 may define an interior volume in which the central rod 203, 303 resides. As also illustrated in FIG. 3, each of the inner tubes 241 and each of the outer tubes 321 may be hollow. In an alternate embodiment, one or more of the inner and/or the outer tubes may not be hollow.

FIG. 9 illustrates the adjustable stop screws on the handle illustrated in FIG. 6A, which may lock the stop 313 in position along slots 347.

After the outer tubes 221, 321 have been expanded to the desired or maximum amount, the handle 205, 305 may be detached from the tubes 201, 301 by cutting the tubes 201, 1 between the clamp and the handle 205 305, and one or more radiation sources, such as one or more radioactive seeds, may be inserted into the hollow interior of one or more of the inner tubes 203, 303 and/or the outer tubes 201, 301 in accordance with a treatment plan. Examples of apparatuses and related processes that may be used in connection with these steps are described in U.S. Provisional Application Ser. No. 60/864,288, entitled “BRACHYTHERAPY DEVICE HAVING SEED TUBES WITH INDIVIDUALLY-SETTABLE TISSUE SPACINGS,” filed Nov. 3, 2006, attorney docket number 63344-045, the entire content of which is incorporated herein by reference.

The components, steps, features, objects, benefits and advantages that have been discussed are merely illustrative. None of them, nor the discussions relating to them, are intended to limit the scope of protection in any way. Numerous other embodiments are also contemplated, including embodiments that have fewer, additional, and/or different components, steps, features, objects, benefits and advantages. The components and steps may also be arranged and ordered differently.

For example, a fluid barrier, such as an expandable sheath, may be placed around the tubes 201, 301 so as to prevent fluid from filling the cage defined by the outer tubes 201 both before and after they are expanded. The interior of this fluid barrier may be filled with air or fluid from an external source. For example, the central rod 203, 303 may have a lumen through which fluid may be delivered from outside of the breast. The fluid may flow into the interior of the fluid barrier through one or more holes in the proximal end of the central rod 203, 303 (not shown).

Although certain mechanical designs have been described to effectuate the bowing of the tubes 201, 303 these are only examples. Any other design may be used instead.

Appropriate apparatuses may also be provided to allow the degree of bowing in the inner tubes 203 and/or the outer tubes 201 to be individually adjusted either before, during or after these tubes are bowed in unison as a result of the rotation of the rotatable ring 111. U.S. Provisional Patent Application Ser. No. 60/864,288, entitled “BRACHYTHERAPY DEVICE HAVING SEED TUBES WITH INDIVIDUALLY-SETTABLE TISSUE SPACINGS,” filed Nov. 3, 2006, attorney docket number 63344-045, sets forth examples of apparatuses that may be used to effectuate such individual adjustments. Any of the apparatuses and methods shown in U.S. patent application Ser. Nos. 11/305,437, entitled “BRACHYTHERAPY APPARATUS,” filed Dec. 16, 2005, attorney docket no. 63344-021, and Ser. No. 11/379,739, entitled “BRACHYTHERAPY APPARATUS FOR ASYMMETRICAL CAVITIES,” filed Apr. 21, 2006, Attorney Docket No. 63344-023, may be used in addition or instead.

Although having thus-far been describe for use in connection with treating a breast, the brachytherapy devices and methods that have been described may also be used to treat other areas of a body, such as the brain or prostrate.

The phrase “means for” when used in a claim embraces the corresponding structures and materials that have been described and their equivalents. Similarly, the phrase “step for” when used in a claim embraces the corresponding acts that have been described and their equivalents. The absence of these phrases means that the claim is not limited to any of the corresponding structures, materials, or acts or to their equivalents.

Nothing that has been stated or illustrated is intended to cause a dedication of any component, step, feature, object, benefit, advantage, or equivalent to the public, regardless of whether it is recited in the claims.

In short, the scope of protection is limited solely by the claims that now follow. That scope is intended to be as broad as is reasonably consistent with the language that is used in the claims and to encompass all structural and functional equivalents.

We claim:

1. A brachytherapy device comprising:
an expandable outer cage configured to expand so as to substantially fill a resected cavity;
an expandable inner cage positioned and configured to expand within the outer cage and configured to receive radioactive material at its perimeter;
an outer cage actuation mechanism configured to cause the outer cage to expand to one of several diameters so as to cause the outer cage to fill a resected cavity having one of several possible diameters; and
an inner cage actuation mechanism configured to cause the inner cage to expand to a fixed diameter that does not vary as a function of the diameter to which the outer cage has been expanded.

2. The brachytherapy device of claim 1 wherein the outer and inner cages each include a plurality of tubes, and wherein each of the tubes have a proximal end and a distal end, wherein the proximal end of all the tubes are affixed to one another.

3. The brachytherapy device of claim 1 wherein the inner cage actuation mechanism includes a carrier configured to engage the distal end of the inner cage, and a stop configured to stop the carrier from moving after the carrier has moved a certain distance.

4. The brachytherapy device of claim 3, wherein the position of the stop is configured to be adjustable.

5. The brachytherapy device of claim 4, further comprising a lead screw configured to adjust the position of the stop.

6. The brachytherapy device of claim 4, further comprising lock screws configured to lock the position of the stop.

7. The brachytherapy device of claim 1 further comprising a compressed spring configured to bias the inner cage distally, and a release mechanism configured to controllably allow the expandable inner cage to expand under the force of the spring.

8. The brachytherapy device of claim 1 wherein the actuation mechanism comprises a rack and gear mechanism configured to urge the expandable outer cage to expand in response to user actuation of the rack and gear mechanism.

9. The brachytherapy device of claim 8 further comprising a thumb wheel configured to cause the outer expandable cage to expand in response to rotation of thumb wheel by a user.

10. The brachytherapy device of claim 9 wherein the thumb wheel is configured to engage a rotatable gear which rotates in response to rotation of the thumb wheel.

11. The brachytherapy device of claim 10 further comprising a rack configured to move distally in response to rotation of the rotatable gear.

12. The brachytherapy device of claim 11 wherein the rack is configured to engage a portion of a carrier which is configured to engage the distal end of the outer cage.

13. The brachytherapy device of claim 1, comprising a plurality of outer tubes configured to have hollow lumens.

14. The brachytherapy device of claim 13 wherein a super-elastic insert is configured to be inserted into the lumen of the hollow lumen.

15. The brachytherapy device of claim 14 wherein the super-elastic insert comprises Nitinol.

16. A brachytherapy for providing brachytherapy comprising:
inserting a brachytherapy device in a resected cavity, the device having an expandable outer cage and an expandable inner cage positioned within the outer cage and configured to receive radioactive material at its perimeter;
expanding the inner and outer cages to a first diameter;
expanding the outer cage to a second diameter that is larger than the first diameter; and
expanding the outer cage to a second diameter that is larger than the first without also expanding the inner cage beyond the first diameter.

17. The brachytherapy of claim 16 wherein the inner cage actuation mechanism includes a carrier configured to engage the distal end of the inner cage, and a stop configured to stop the carrier from moving after the carrier has moved a certain distance.

18. The brachytherapy of claim 17 wherein the position of the stop is configured to be adjustable.

19. The brachytherapy of claim 16 further comprising a compressed spring configured to bias the inner cage distally, and a release mechanism configured to controllably allow the expandable inner cage to expand under the force of the spring.

20. The brachytherapy of claim 16 wherein the device comprises a rack and gear actuation mechanism configured to urge the expandable outer cage to expand in response to user actuation of the rack and gear mechanism.

21. The brachytherapy of claim 16 wherein the device comprises a thumb wheel configured to cause the expandable cage to expand in response to rotation of the thumb wheel by a user.

22. The brachytherapy of claim 16 wherein the device comprises a plurality of outer tubes configured to have hollow lumens.

23. The brachytherapy device of claim 22 wherein a super-elastic insert is configured to be inserted into the lumen of the hollow lumen.

24. A brachytherapy device comprising:
an expandable cage configured to receive radioactive material at its perimeter;
a compressed spring configured to cause the expandable cage to expand; and
a release mechanism configured to controllably allow the expandable cage to expand under the force of the spring.

25. The brachytherapy device of claim 24, further comprising a stop configured to stop the cage from expanding under the force of the compressed spring at a pre-determined diameter.

26. The brachytherapy device of claim 25 further comprising a carrier configured to apply force to an end of the expandable cage and to engage the stop.

27. The brachytherapy device of claim 25 wherein the position of the stop is configured to be adjustable.

28. The brachytherapy device of claim 24 wherein the device comprises a thumb wheel configured to cause the expandable cage to expand in response to rotation of the thumb wheel by a user.

29. The brachytherapy device of claim 24 wherein the device comprises a plurality of inner tubes configured to have hollow lumens.

30. A brachytherapy device comprising:
an expandable cage configured to receive radioactive material at its perimeter; and
a rack and pinion mechanism configured to urge the expandable cage to expand in response to user actuation of the rack and pinion mechanism.

31. The brachytherapy device of claim 30 wherein the pinion comprises one or more gears, wherein rotation of a gear moves the rack in a longitudinal direction.

32. The brachytherapy device of claim 31 wherein the rack is configured to cause an expandable cage carrier to move longitudinally and,
wherein the expandable cage carrier is configured to cause expansion of the expandable cage.

33. A brachytherapy device comprising: an expandable cage configured to receive radioactive material at its perimeter; and
a thumb wheel configured to cause the expandable cage to expand in response to rotation of the thumb wheel by a user.

34. The brachytherapy device of claim 33, further comprising a handle having a casing and wherein the thumb wheel is configured to partially protrude from the casing of the handle.

35. The brachytherapy device of claim 33 wherein the thumb wheel is configured to rotate in a forward and backward direction.

36. The brachytherapy device of claim 33 wherein the thumb wheel comprises a first gear.

37. The brachytherapy device of claim 36 wherein rotation of the first gear is configured to cause a rack to move in a longitudinal direction,
wherein the rack is configured to cause an expandable cage carrier to move longitudinally and,
wherein the expandable cage carrier is configured to cause expansion of the expandable cage.

38. A brachytherapy device comprising:
an expandable cage configured to expand within a resected cavity;
and a super-elastic alloy configured to collapse the cage when an actuation mechanism is released;
wherein the device comprises apparatus configured to receive radioactive material.

39. The brachytherapy device of claim 38 wherein the device comprises a plurality of outer tubes configured to have hollow lumens and,
wherein a super-elastic alloy is configured to be inserted into the lumen of the hollow lumen.

40. The brachytherapy device of claim 39 wherein the super-elastic alloy is in the shape of a wire.

41. The brachytherapy device of claim 39 wherein the super-elastic alloy is Nitinol.

42. The brachytherapy device of claim 38 comprising an expandable outer cage and expandable inner cage positioned and configured to expand within the outer cage and configured to receive radioactive material at its perimeter.

43. A brachytherapy device comprising:
an expandable outer cage configured to expand so as to substantially fill a resected cavity;
an expandable inner cage positioned and configured to expand within the outer cage and configured to receive radioactive material at its perimeter;
an actuation mechanism configured to cause the outer cage to expand to one of several diameters so as to cause the outer cage to substantially fill a resected cavity having one of several possible diameters and to cause the inner cage to expand simultaneously with the outer cage and at a diameter that is less than the diameter of the outer cage.

44. The brachytherapy device of claim 43 wherein the outer and inner cages each include a plurality of tubes, and wherein each of the tubes have a proximal end and a distal end, wherein the proximal end of all the tubes are affixed to one another.

45. The brachytherapy device of claim 44 wherein the tubes of the inner cage are configured to be engaged by an inner tube carrier and the tubes of the outer cage are configured to be engaged by an outer tube carrier and wherein each carrier comprises an angular slot.

46. The brachytherapy device of claim 45 wherein the inner and outer tube carriers are configured to be connected by a hair pin connector at the angular slots.

47. The brachytherapy device of claim 43 wherein the inner and outer actuation mechanism comprises a rack and pinion mechanism configured to urge the expandable inner and outer cages to expand in response to user actuation of the rack and pinion mechanism.

48. The brachytherapy device of claim 47 wherein the rack is configured to move the outer tube carrier in a longitudinal direction.

49. The brachytherapy device of claim 47 wherein the rack is configured to move the inner tube carrier in a longitudinal direction via a hair pin connector.

50. The brachytherapy device of claim 49 wherein the distal end of the hair pin connector is attached to a bracket.
